(19) United States
(12) Patent Application Publication
(10) Pub. No.: US 2010/0081930 A1
(43) Pub. Date: Apr. 1, 2010

(54) Systems and Methods for the Display of Ultrasound Images

(75) Inventor: Lee Dunbar, Bothell, WA (US)

Correspondence Address: SonoSite, Inc. / Fulbright & Jaworski, L.L.P., 2200 Ross Avenue, Suite 2800, Dallas, TX, 75201, US

(73) Assignee: Sonosite, Inc., Bothell, WA (US)

(21) Appl. No.: 12/240,634

(22) Filed: Sep. 29, 2008

(51) Int. Cl. A61B 8/00

(52) U.S. Cl. 600/437

(57) ABSTRACT

A method of real time sonographing that includes concurrently displaying a real time ultrasound image and a patient record in a sonographer's paracentral vision. An ultrasound device including a display adapted to concurrently project a real time ultrasound image and a patient record. A method that includes displaying a non-real time ultrasound image and a patient record in a sonographer's paracentral vision. An ultrasound device including a display adapted to project a non real time ultrasound image and a patient record.

### TECHNICAL FIELD

This disclosure relates to ultrasound imaging and more particularly to systems and methods for displaying ultrasound images and patient records.

### BACKGROUND OF THE INVENTION

Ultrasound is sound with a frequency greater than 20,000 Hz—approximately the upper limit of human hearing. Humans therefore usually cannot hear ultrasound. Ultrasound is utilized for imaging in many different fields. To create an image, ultrasound is made to penetrate a medium and the reflections from the medium are measured and displayed to represent the internal portions of the medium. For example, ultrasound scanning or sonography involves exposing a part of a person's body to ultrasound. The different components of that part of the body reflect the ultrasound differently. Therefore, the reflected ultrasound creates an image of the different components such as organs, tissue, blood vessels etc. One benefit of ultrasound over other imaging techniques, such as x-rays, is that it is captured and observed in real-time. An example of a handheld ultrasound device is disclosed in U.S. Pat. No. 5,722,412, the disclosure of which is incorporated herein by reference.

A sonographer is a person who operates ultrasonic imaging devices. Typically, in the medical field, ultrasound devices display ultrasound images with the name of the patient and institution where the ultrasound image is being taken. The sonographer usually accesses the patient's medical record from another screen of the ultrasound device, or from another computer's screen or from paper. Additionally, the sonographer observes data from the ultrasound image and manages a patient record on another screen of the ultrasound device, or on another computer's screen or on paper. This process where the sonographer has to direct attention from one medium to another to interpret the ultrasound image and then manage a patient record is time consuming and sometimes confusing. Moreover, the sonographer is subject to making mistakes using this method because it requires the sonographer to memorize and process information as the sonographer moves from one screen to the next or to paper. In sum, current ultrasound devices causes a sonographer to rely heavily on memory or an intermediate step to process a real time ultrasound image and to manage a patient's medical record, which in turn affects the sonographer's efficiency and accuracy.

### BRIEF SUMMARY OF THE INVENTION

The present invention is directed to a system and method which improves the efficiency of a sonographer by facilitating less reliance on memory as the sonographer mentally processes a real time ultrasound image during an ultrasound examination of a patient and manages the patient's medical record. Managing a patient record includes generating, creating, updating, revising and reviewing the patient record. One embodiment of the invention accomplishes this by presenting the ultrasound image and the technical data concurrently in a sonographer's paracentral vision.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows an example of the work flow of an ultrasound examination;

FIG. 2 shows a prior art illustration of how sonographers currently use ultrasound technology;

FIG. 3 is one embodiment of the invention;

FIGS. 4A-4E are processes of embodiments of the invention;

FIG. 5 is a process of one embodiment of the invention;

FIG. 6 is a process of one embodiment of the invention; and

FIG. 7 is a block diagram of one embodiment of the invention.

### DETAILED DESCRIPTION OF THE INVENTION

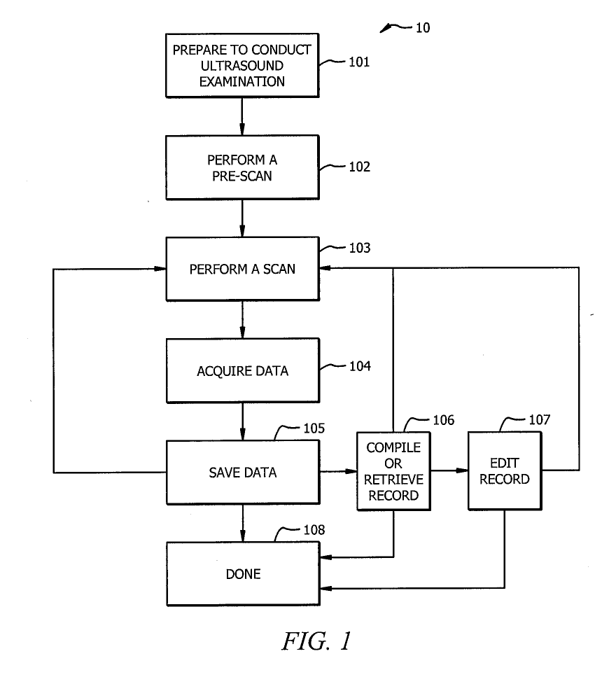

FIG. 1 shows work flow 10 of a sonographer doing an ultrasound examination of a patient. In process 101, the sonographer prepares to conduct the examination. This may include entering patient identification and the procedure being performed into the database of an ultrasound device. Preparation may also involve applying a gel to the area of the body to be scanned. In process 102, the sonographer uses the transducer—the component of the ultrasound machine that emits the ultrasound—to prescan a part of the patient's body by moving the activated transducer against the body. In process 103, the sonographer may then apply more gel to the relevant part of the body and performs a complete scan of the relevant part of the body.

As the sonographer views the ultrasound images in process 103, the sonographer may determine that the ultrasound image on the screen shows something of interest. In process 104, the sonographer freezes the ultrasound image and measures portions of the ultrasound image. In process 105, the sonographer enters these measurements or other information into a database using another screen or paper. The database is stored on a computer readable medium of the ultrasound device. Often times, after the sonographer saves the measurements or other information in process 105, the sonographer repeats process 103 and identifies a measurement better than the previous one, or identifies another point of interest. In such a scenario, the sonographer then repeats the acquisition and saving of data in processes 104 and 105 and may replace or augment previously saved data or save the new data as additional data.

In process 106, the sonographer may use measurements or other information from the ultrasound image to compile a patient record. Alternatively, in process 106, the sonographer may retrieve a previously compiled patient record. In process 107, the sonographer may change edit or delete the previously stored patient record and then return to do further scans, data acquisition and saving in processes 103, 104 and 105, respectively. The sonographer may repeat processes 103-105 several times to refine the patient record. One can see, therefore, that the sonographer necessarily and continuously moves back and forth between screens or between screen and paper to view an ultrasound image and manage the patient record.

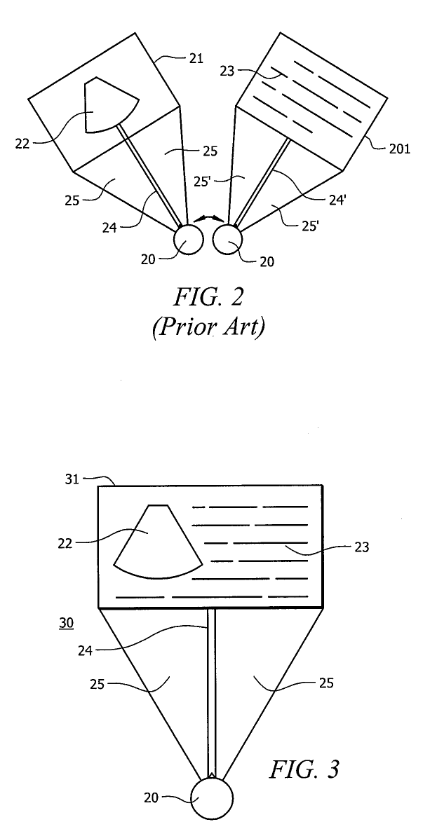

FIG. 2 shows how, in current ultrasound imaging technology, sonographer 20 must move his or her focus and attention from screen 21 to screen 201 to perceive ultrasound image 22 on screen 21 and then to see and manage patient record 23 on screen 201. It should be noted that, instead of a screen, patient record 23 may be on paper. If sonographer 20 is viewing screen 22 at any time, screen 22 will be within sonographer's central area of gaze 24 or in sonographer's paracentral vision 25. Paracentral vision 25 is the area immediately adjacent to central area of gaze 24. Thus, for example, it is possible for one to read words in one's paracentral vision but not possible to do so for views outside one's paracentral vision—peripheral vision. Significantly, screen 23 is in sonographer's peripheral view when sonographer 20's central area of gaze is on screen 21 and thus patient record 23 on screen 201 cannot be read without sonographer 20 turning his or her head clockwise and thereby changing sonographer 20's central area of gaze to central area of gaze 24¹. Correspondingly, sonographer 20's new paracentral vision is paracentral vision 25¹.

In sum, if sonographer 20 is viewing screen 21 and then wishes to view screen 201, sonographer 20 must turn his or her head clockwise to do so. After viewing screen 201 if sonographer 20 wishes to again view screen 21, sonographer 20 must move his or her head counter clockwise or up or down. Currently, sonographers have to do this repeatedly and it is not only physically tiring but also mentally challenging. Significantly, for sonographer 20 to mentally relate the information from ultrasound image 22 with patient record 23 sonographer 20 must remember the information perceived in the screen not being viewed. In other words, when sonographer 20 views screen 21, sonographer 20 must remember details shown on 201 and vice versa. This reliance on memory is also necessary if ultrasound image 22 and 23 are displayed consecutively on one screen, say, screen 21.

FIG. 3 illustrates one embodiment 30 of the invention—display 31 that allows sonographer 20 to avoid moving his or her head back and forth between an ultrasound image and a screen where sonographer 20 manages a patient record, such as screen 21 and 201. An ultrasound device display 31 displays, concurrently, ultrasound image 22 and patient record 23 in paracentral vision 25 of sonographer 20. In some embodiments, the concurrent display of an ultrasound image 22 and patient record 23 is presented in the central area of gaze 24.

Concurrently displaying a real-time ultrasound image 22 and patient record 23 in a sonographer's paracentral vision or center of gaze allows the sonographer to view ultrasound image 22 and patient record 23 without having to access another display, flick through different screens on a display or move one's head. Thus, display 31 enables sonographer 20 to avoid the effort and time it would take to access separate screens or media for an ultrasound image and a patient record. This makes it easy for sonographer 20 to analyze information from ultrasound image 22 and to manage patient record 23 at the same time. The sonographer may enter data to manage a patient record by any method, such as by a touch screen feature of display 31 or by a keyboard.

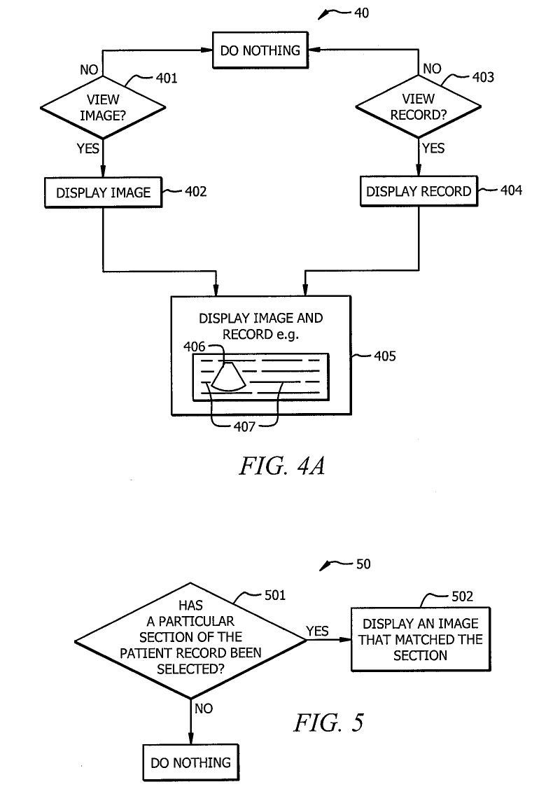

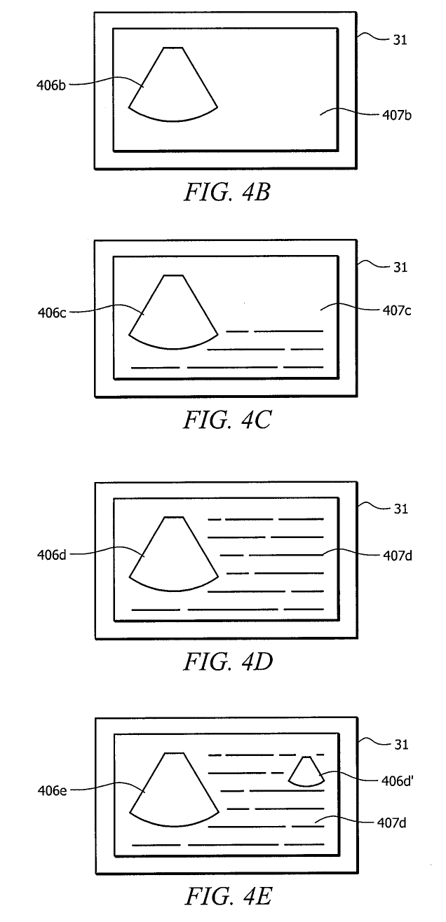

FIG. 4A shows the process involved in one embodiment 40 of the invention where the ultrasound device allows the sonographer to determine in processes 401 and 404 whether to view, concurrently, the image and the patient record. If the sonographer wishes to view both, concurrently, then processes 402 and 404 display ultrasound image 406 and patient record 407 at the same time. In other words, this embodiment of the invention achieves a parallel operation as opposed to the prior art which shows a sequential operation. Thus, in process 405, ultrasound image 406 and patient record 407 are displayed together on a screen so as to fit into a sonographer's paracentral vision or central area of gaze while doing an ultrasound examination. It should be noted that embodiment 40 could involve the concurrent display of any combination of: a real time ultrasound image, a non real time ultrasound image, a current patient record and a previous patient record.

FIGS. 4B-4E shows how a sonographer generates a patient record 407 using embodiments of the invention. In this example, the sonographer is a medical doctor. The doctor performs a medical procedure on a patient that includes sonographing the chest of a patient. Thus, the doctor may start sonographing the left of the patient's chest and display 31 shows a real time ultrasound image of the left of the patient's chest 406b. Because in this example the patient is new, patient record 407b has no data in it.

As the doctor observes real time ultrasound image 406b, however, the doctor is able to record data in patient record 407. Therefore, by the time the doctor sonographs the center of the patient's chest and display 31 shows center chest real time ultrasound image 406c, the doctor has recorded clinical information to patient record 407c during the current sonographic session.

The doctor then proceeds to sonograph the right of the patient's chest to display right chest real time ultrasound image 406e. As real time ultrasound image 406e is displayed, the doctor completes patient record 407d for this medical procedure. The doctor may save the patient record 407d to a storage device, such as storage device 72 in FIG. 7. In one embodiment, the doctor may save ultrasound images from real time ultrasound images 406b-406d.

If the patient returns to the doctor, say, months later the doctor may repeat the same procedure by recalling previously stored patient record 407d. In one embodiment of the invention, patient record 407d may include a non-real time ultrasound image 406d¹ which was saved from the previous real time ultrasound image 406d.

FIG. 5 shows process 50 of one embodiment of the invention that correlates the ultrasound image with a section of a patient's record. Process 501 determines whether a particular section of a patient record has been selected for example by a mouse cursor being positioned over that section of the patient record. If a particular section of the patient record has been selected, process 502 automatically displays the ultrasound image that matches the particular section. To facilitate this type of correlation, the software program for the ultrasound device allows the sonographer to store information, in relation to a particular ultrasound image, in a database on a storage medium which can be within the ultrasound device. Alternatively, the ultrasound device's software program makes this correlation automatically based on the ultrasound image displayed when the sonographer initially recorded that section of the patient record.

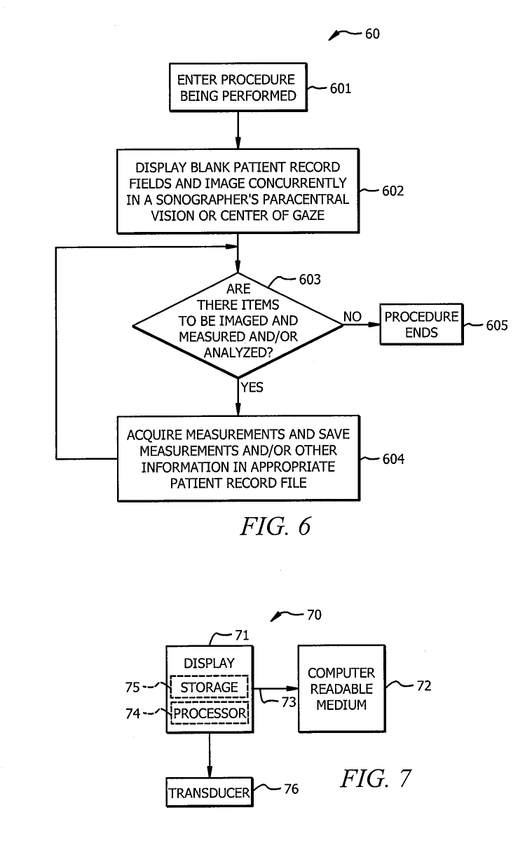

FIG. 6 is a flow diagram 60 showing the process of performing an ultrasound examination that involves preparing a patient record using one embodiment of the invention. In process 601, the sonographer enters the name of the sonographic procedure into the database of the ultrasound device. In process 602, the ultrasound device concurrently displays, in a sonographer's paracentral vision or center of gaze, an ultrasound image and fields, which are capable of accepting data for a patient's record. In process 603, the ultrasound device determines (either by asking the sonographer or by use of an internal program) whether there are items to be imaged and measured. If there are, process 604 instructs the user to acquire and save the measurements and/or other information. Process 604 is repeated as many times as is necessary. If there are no items to be imaged then process 605 ends the procedure.

FIG. 7 shows a block diagram system 70 that represents one embodiment of the invention. Display 71 is capable of concurrently projecting a real time ultrasound image and a patient record. Display 71 is connected to computer readable medium 72 by communication link 73. Computer readable medium 72 is capable of storing, among other things, software for operating the ultrasound system and a patient record projected on display 71. Communication link 73 may be any type of communication link including wire or wireless. In one embodiment, processor 74 controls the flow and processing of data to/from transducer 76 and to/from computer readable medium 72 and storage 75. Note that in some embodiments, elements 71, 72 and 76 can be in a common housing, or element 72 could be partially in the housing and partially remote therefrom. In one embodiment, system 70 may be a review station. In this embodiment, display 71 concurrently displays an ultrasound image from a previously conducted ultrasound session and a previously stored patient record. The ultrasound image from the previous session and the previous patient record may be retrieved from storage on computer readable medium 72.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of real time sonographing, said method comprising:
concurrently displaying a real time ultrasound image and a patient record in a sonographer's paracentral vision.

2. The method of claim 1 wherein said displaying of said ultrasound image and said patient record is within said sonographer's center of gaze.

3. The method of claim 1 wherein said displaying is done by a single electronic screen of an ultrasound device.

4. The method of claim 1 wherein said patient record is created during said real time sonographing.

5. The method of claim 1 wherein said patient record is updated during said real time sonographing.

6. The method of claim 1 wherein said patient record was stored, prior to said real time sonographing, in a computer readable medium.

7. The method of claim 1 further comprising:
said sonographer managing said patient record.

8. The method of claim 1 further comprising:
identifying information from said ultrasound image; and
correlating said information with said ultrasound image while both said ultrasound image and said correlated information are displayed concurrently.

9. The method of claim 8 wherein said correlation involves displaying said ultrasound image when said information is selected.

10. The method of claim 8 wherein said correlation involves displaying said information when said ultrasound image is selected.

11. An ultrasound device comprising:
a display adapted to concurrently show a real time ultrasound image and a patient record; and
a processor adapted for operating said ultrasound device, said processor comprising computer readable medium for storing said patient record.

12. The ultrasound device of claim 11 wherein said display is an electronic screen.

13. The ultrasound device of claim 12 wherein said electronic screen is a touch screen.

14. The ultrasound device of claim 11 wherein said ultrasound image is an image of at least part of said patient's body and said concurrently displayed patient record comprises data from a current sonographic session.

15. The ultrasound device of claim 11 wherein said ultrasound image is an image of at least part of said patient's body and said concurrently displayed patient record comprises retrieved data.

16. The ultrasound device of claim 15 wherein said retrieved data is selected from: a previous patient record, an ultrasound image from a previous sonographic session.

17. An ultrasound system comprising:
means for concurrently showing a real time ultrasound image and a patient record; and
means for storing said patient record.

18. The ultrasound system of claim 17 wherein said means for concurrently showing is an electronic screen.

19. The ultrasound system of claim 17 wherein said electronic screen is a touch screen.

20. The ultrasound system of claim 17 wherein said ultrasound image is an image of a patient and said concurrently displayed patient record comprises data from a current sonographic session.

21. The ultrasound system of claim 17 wherein said ultrasound image is an image of a patient and said concurrently displayed patient record comprises retrieved data.

22. The ultrasound system of claim 21 wherein said retrieved data is selected from: a previous patient record, an ultrasound image from a previous sonographic session.

23. A method comprising:
concurrently displaying a non-real time ultrasound image and a patient record in a user's paracentral vision.

24. The method of claim 23 wherein said displaying of said ultrasound image and said patient record is within said user's center of gaze.

25. The method of claim 23 wherein said displaying is done by a single electronic screen of an ultrasound device.

26. The method of claim 23 wherein said non-real time ultrasound image and said patient record are retrieved from a computer readable medium's storage.

27. The method of claim 23 further comprising:
managing said patient record.

28. An ultrasound device comprising:
a display adapted to concurrently show a non-real time ultrasound image and a patient record; and
a processor adapted for operating said ultrasound device, said processor comprising computer readable medium for storing said non-real time ultrasound image and said patient record.

29. The ultrasound device of claim 28 wherein said display is an electronic screen.

30. The ultrasound device of claim 29 wherein said electronic screen is a touch screen.

31. An ultrasound system comprising:
means for concurrently showing a non-real time ultrasound image and a patient record; and
means for storing said patient record.

32. The ultrasound system of claim 31 wherein said means for concurrently showing is an electronic screen.

33. The ultrasound system of claim 31 wherein said electronic screen is a touch screen.
